(19) United States
(12) Patent Application Publication
(10) Pub. No.: US 2015/0275114 A1
(43) Pub. Date: Oct. 1, 2015

(54) PROCESS FOR CO-PRODUCTION OF BIO-ENERGY AND PRODUCTS FROM INTEGRATED CONVERSION OF BIOMASSES AND MUNICIPAL WASTES

(71) Applicant: SEA MARCONI TECHNOLOGIES DI VANDER TUMIATTI S.a.s., Collegno (Torino) (IT)

(72) Inventors: Vander Tumiatti, Rosta (Torino) (IT); Francesco Lenzi, Bibbiena-Frazione Soci (Arezzo) (IT); Michela Tumiatti, Torino (IT)

(21) Appl. No.: 14/434,709

(22) PCT Filed: Oct. 11, 2013
(86) PCT No.: PCT/EP2013/071320

§ 371 Date: Apr. 9, 2015

(30) Foreign Application Priority Data
Oct. 12, 2012 (IT) TO2012A000894

(51) Int. Cl. C10L 3/00; A01G 9/18; C02F 3/32; A01G 9/14; C12P 1/04; C05F 9/04

(52) U.S. Cl. CPC C10L 3/00; C12P 1/04; C05F 9/04; C02F 3/322; A01G 9/14; A01G 9/18; C10L 2290/26

(57) ABSTRACT

A process of using algal and lignocellulosic biomasses deriving from the exploitation of the by-products generated by primary conversion systems, digestate, biochar, water, CO2 and nitrogenous and sulphurised compounds and ashes as nutrients for the production of vegetable organisms by intensifying the photosynthesis processes for the treatment of emissions and the sequestration of CO2; feeding a substratum to a press-extrusion system so that it is sub-divided into a liquid organic fraction and an energetic dry fraction; using a liquid organic fraction as feed for a biogas generation system by bacterial fermentation under anaerobic conditions; using the energetic dry fraction as feed for a synthesis gas generation system by thermo-chemical conversion; wetland bio-filtering water obtained as by-product from the biogas and syngas generation system, obtaining purified water and primary biomass; using biogas and/or synthesis gas produced by a co-generation system and/or biofuels deriving from biogas and/or synthesis gas to feed vehicles.

### FIELD OF APPLICATION

This invention relates to a process for the co-production of bio-energy and products by means of the integrated conversion of biomasses, municipal wastes and/or carbonaceous matrices.

Within this description of the invention and its field of application, the main terms are defined in WO2012/085880.

### STATE OF THE ART

The conversion processes of municipal wastes, biomasses and/or carbonaceous matrices must comply with the prescribed requisites within the local and European normative framework. Directive 2008/98/EC prescribed in Art. 16 the “principles of self sufficiency and proximity” with the implementation, in conjunction with other Member States should this be necessary or appropriate, of appropriate measures for the creation of an integrated and appropriate network of systems for the disposal of wastes and systems for the recovery of unsorted municipal wastes coming from domestic collection, as well as the cases where such collection includes such wastes coming from other producers, taking into account the best available techniques (BAT) and the best environmental practices (BEP).

In Italy, the Decree of 29 Jan. 2007 prescribes the Guidelines for the identification and the use of the best available techniques on the subject of the management of wastes, for the activities listed in enclosure I of legislative decree of 18 Feb. 2005, no. 59. At the level of UNCCD (United Nations Convention to Combat Desertification—2012) the critical factors to prevent and mitigate desertification are also focused.

Within the State of the Technique the patents, the systems and the processes indicated below are significant.

In EP-A-1 354 172 it is mainly claimed a reactor equipped with screw into which thermically conductive bodies are sent together with the process carbonaceous matrix, designated as HALOCLEAN®. These bodies are metal, ceramic spheres and SiC. Mostly they have the function of keeping the internal surface of the reactor and the screw clean. The HALOCLEAN® process has been indicated as BAT for the conversion and/or decontamination of materials and wastes contaminated by PCBs (Italian Ministry of Environment with M.D. 29.01.2007). In its applications (i. e. WEEE, biomasses etc.) Haloclean® is considered the pioneer of “Intermediate Pyrolysis”.

In WO2009138746 A1 it is described a treatment process based upon biomasses and in particular algae.

In Patent Application TO2008A000394 it is described a system for the stabilisation of organic material coming from municipal solid wastes including a mixing silo for the homogenisation and a station for an aerobic digestion for the degradation in the absence of oxygen by the action of different groups of micro-organisms made almost exclusively by anaerobic and facultative bacteria with subsequent production of biogas.

PCT WO 2012/085880 A2 is focused upon a modular system where the base module is made of a rotating reactor with a fixed casing, an actuating system, the presence of thermally conductive bodies and a heating/cooling group. The base module is functionalised and configured in series or parallel to provide the required conversion operational conditions.

Patent Application ITTO20100192 describes a system including a frame and a horizontal drum supporting a triad of perforated cylindrical squeezing chambers open at their opposite ends for the separation by pressing extrusion of the wet fraction and the dry fraction deriving from solid municipal wastes.

Patent Application TO2011A000873 describes the specific use for the application on vegetable organisms (i.e. biomasses, fruit and/or ornamental plants, cereals, algae etc.) of which the growing conservation, protection and/or disinfection is promoted by a functionalised nanosponge, consisting of a reticulated cyclodextrine containing at least one functionalising agent such as a micro element, an active principle and/or a magnetic material.

Historically the disposal of solid municipal wastes has mostly taken place by dumping in controlled landfills. The normative evolution of the sector has stimulated new technological solutions based upon selective collection and recovery of materials as well the combustion by means of high temperature controlled incineration with the recovery of electric and thermal energy (reference Ministry of Environment Decree 29.01.2007).

An additional orientation is the valorisation of wastes with the transformation into SSF (Secondary Solid Fuels—UNI CEN/TS 41359) for use in industrial cycles such as, for example, cement factories (Report “Potentiality and benefits of Secondary Solid Fuels (SSF) in industries—NE Nomisma Energia Srl—December 2011).

Waste to energy plants are justifiable only for relatively large collection communities (basins) and they determine important logistic implications, use of the territory and environmental impact as well as significant CO₂, micro pollutants emissions (i.e. POPs such as PCDD-Dioxins and PCDF-Furans etc.) and dusts, ashes and the production of solid residues (example up to 26% and beyond of the initial weight to be sent to landfills).

The most recent orientations formalised by the Summit Rio+20 (2012) on sustainable development and climate changes focus on Green Economy solutions in terms of technological, economical, environmental and social terms. The implementation of these guidelines find a support in the European projects “Horizon 2020” and at Italian level in actions oriented toward the development of “Smart Cities, Smart Communities & Smart Grids”.

In a global scenario of sustainability climate conditions must also be considered (i.e. temperate, extreme cold, extreme hot climates etc.) and a large variety of specific, social-economical and territorial characteristics (i.e. low density of inhabitants concentrated in small urban agglomerates, risks of erosion and desertification, water shortages, dryness etc.).

The single techniques and/or treatment processes of municipal wastes and biomasses do not solve the current criticalities lacking one or more requisites of sustainability.

The water contained by the waste and/or biomass is a precious resource to be valorised being always intrinsically available also in zones with a water deficiency (i.e. desert zones etc.) or under seasonal or permanent dryness conditions.

The geo-climatic and social-economical conditions are important variables to be considered since maximum and minimum temperatures, wind profile, nearby population density and other specific conditions are important factors for the characteristics of the initial wastes, the building and performance features of the conversion systems as well as the justification and the triggering promotion of virtuous circuits to fight dryness, erosion of soil and desertification for the local sustainable development servicing communities.

The wastes and biomasses produced by the local community must become a resource and an opportunity for the widespread sustainable development for the production of bioenergy and products in an efficient, economic, safe and socially acceptable manner, preventing smelly emissions and the NIMBY syndrome.

### SUMMARY OF THE INVENTION

The object of this invention is to provide a co-production process of bioenergy and products deriving from the integrated conversion of biomasses, municipal wastes and/or carbonaceous matrices and a system for the operation of such process in a sustainable manner also for small sizes (≦25.000 t/y) facilitating social acceptability. This is achieved by basing upon highly integrated and flexible technologies and processes as described in the field of application, without the critical factors typical of the known systems and processes.

This object is achieved thanks to a process having the features indicated in claim 1 below. The favourite features of the process of the invention are indicated in the dependent claims 2 through 14.

In detail, this invention is embodied in a process for the recovery and valorisation of a substratum including biomasses, municipal wastes and/or carbonaceous matrices encompassing the following phases:
feeding the said substratum to a press-extrusion system possibly provided of separation means so that it is sub-divided into a liquid fraction and a dry fraction,
using the said liquid fraction as feed for a biogas generation system by means of bacterial fermentation under anaerobic conditions,
using the said dry fraction as feed for a rotary reactor inside a fixed shroud enveloping it, into which a pyro-gasification is carried out at a temperature included between 500 and 1000° C. with the generation of synthesis gas,
phytodepuration bio-filtration of the water resulting as by-product from the biogas generation system, obtaining purified water and primary biomass,
using the biogas and/or synthesis gas produced for the production of thermal, electrical and/or mechanical energy.

Profitably, the digestate obtained as by-product by the biogas generation system is fed to the press-extrusion system together with said substratum.

Profitably, the said purified water is used to irrigate cultivations in greenhouses thermally controlled by means of the thermal energy produced by the combustion of said biogas and/or synthesis gas.

### SHORT DESCRIPTION OF THE FIGURES

Advantages and features of this invention shall be evident from the detailed description to follow, provided as a non-limiting example, with reference to the enclosed drawings where:

FIG. 1 is a schematic representation of the system of the invention in which the blocks represent the techniques and the apparatuses with a strong integration (cluster) making the functional units,

FIG. 2 is a schematic representation of the arrangement plan and a possible configuration of the functional units,

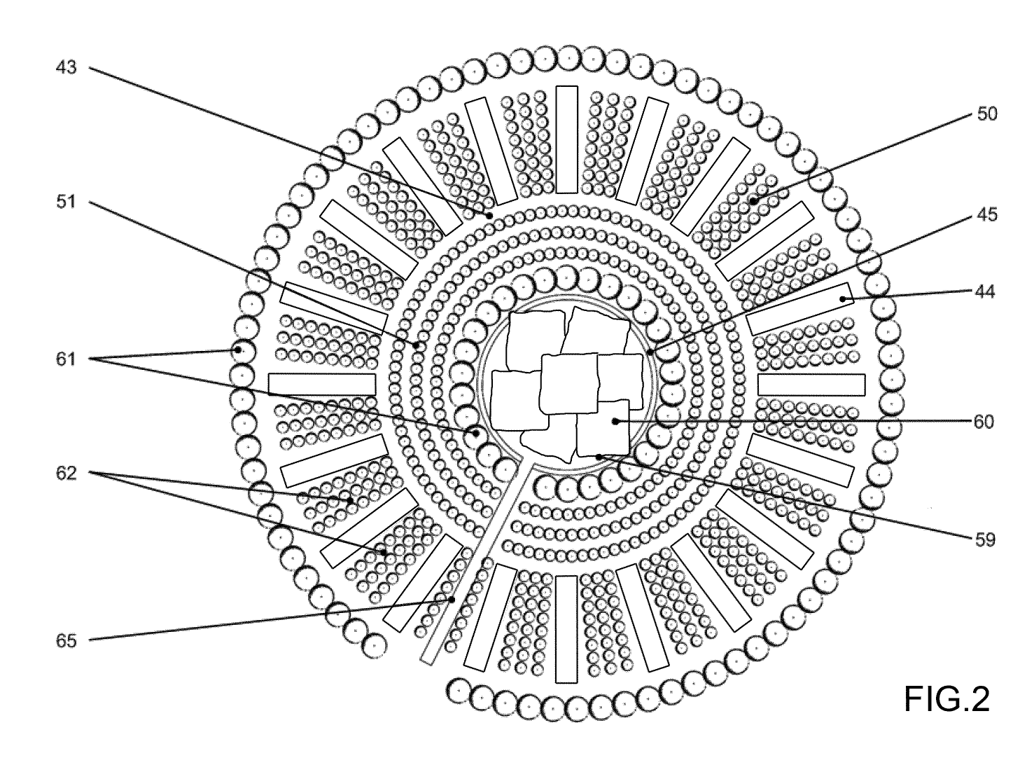

FIG. 3 is a schematic representation of the vertical section arrangement and a possible configuration of the functional units,

FIG. 4 is a schematic representation of the mass and energy balance in the case of integrated conversion of Municipal Solid Wastes (MSW),

FIG. 5 is a schematic representation of the mass and energy balance in the case of Organic Fraction of Municipal Solid Waste (OFMSW).

Shown below are listed, with a progressive numbering, the systems, apparatuses, devices and matrices to be converted, derived products and energy, of the system of the invention:
1. initial carbonaceous matrices particularly deriving from municipal solid wastes
2. initial carbonaceous matrices of biogenic origin
3. reception
4. stocking in confined and protected areas
5. selective separation of ferrous, non-ferrous metals, glass and possible inerts
6. ferrous and non-ferrous metals, inerts
7. press-extrusion
8. organic liquid fraction (OLF)
9. energetic dry fraction (EDF)
10. dehumidification apparatus of the energetic dry fraction
11. separation of plastics
12. plastics
13. intensification apparatus by means of selective hyper-dynamic cavitation
14. intensification apparatus by means of functionalised nanosponges
15. biochemical conversion system for biogas
16. sound apparatus for the suppression of foams
17. apparatus for the treatment, desulphurisation and dehumidification of the biogas
18. biogas
19. biogas powered co-generator
20. water/solid separation from digestate
21. water treatment from biogas system
22. solid from digestate
23. quality digestate
24. recovery and treatment of drying water
25. recovered water
26. water purification apparatus
27. drinking water
28. syngas thermo-chemical conversion system
29. apparatus for the treatment, desulphurisation and dehumidification of the syngas
30. apparatus with solar concentration panels
31. syngas
32. syngas powered co-generation
33. ashes
34. biochar
35. electric energy
36. thermal energy
37. CO₂ and/or nitrogenous and sulphurised compounds
38. apparatus for the extraction of metals from ashes
39. vitrification apparatus for residual solids
40. inerts for construction or landfill
41. apparatus for the manufacturing of multi-wall carbon nanotubes
42. multi-wall carbon nanotubes
43. Smart Farm with intensified greenhouses and open field selective cultivation
44. advanced intensified greenhouses
45. distribution apparatus for irrigation waters
46. distribution apparatus of gaseous matrix enriched with nutrients such as CO₂ and/or nitrogenous and sulphurised compounds to intensify the process
47. semi-transparent thin photovoltaic system
48. apparatus for cycles of high efficiency natural and/or artificial lighting for greenhouses
49. apparatus for electro bio-stimulation for greenhouses
50. open field dedicated cultivation for the production of high yield primary biomass
51. phytodepuration (constructed wetland)
52. production of algal biomass with phytodepuration of gaseous emission and intensification of CO₂
53. functionalised nanosponges for the dispensing of micro-element, nutrients, active principles and disinfectants
54. biofuel production system
55. delignification apparatus of the lignocellulosic and/or algal biomass for bio ethanol
56. apparatus for the intensification of the extraction of oleous fraction from algal biomass by means of hyper-dynamic selective cavitation
57. biofuel and algal biomass
58. Smart Dome architectural structure for the protection of the systems zone
59. tension protection structure for systems zone
60. semi-transparent thin-film photovoltaic system
61. perimeter vegetable anti-wind barrier
62. intercrops production
63. electrical infrastructure (LV/MV cabin) and connection to Smart Grid
64. semantic interface for diagnostic and prognostic coverage
65. access infrastructure to the systems zone infrastructures
66. control, monitoring, intelligent local remote and integrated supervision module of operational performances (functional and environmental) as well as advanced diagnostic procedures for the management of the life cycle of the systems, units and vital components, E-learning and E-maintenance

### DETAILED DESCRIPTION OF THE INVENTION

This invention relates to a process for the co-production of bio-energy and products deriving from the integrated conversion of biomasses, municipal wastes and/or carbonaceous matrices and a system for the performance of such process capable of maximising the quantity of energy, products and reusable substances recovered from the latter minimising, if not totally eliminating, the non-recoverable residue in compliance with what indicated in the field of application and in its object.

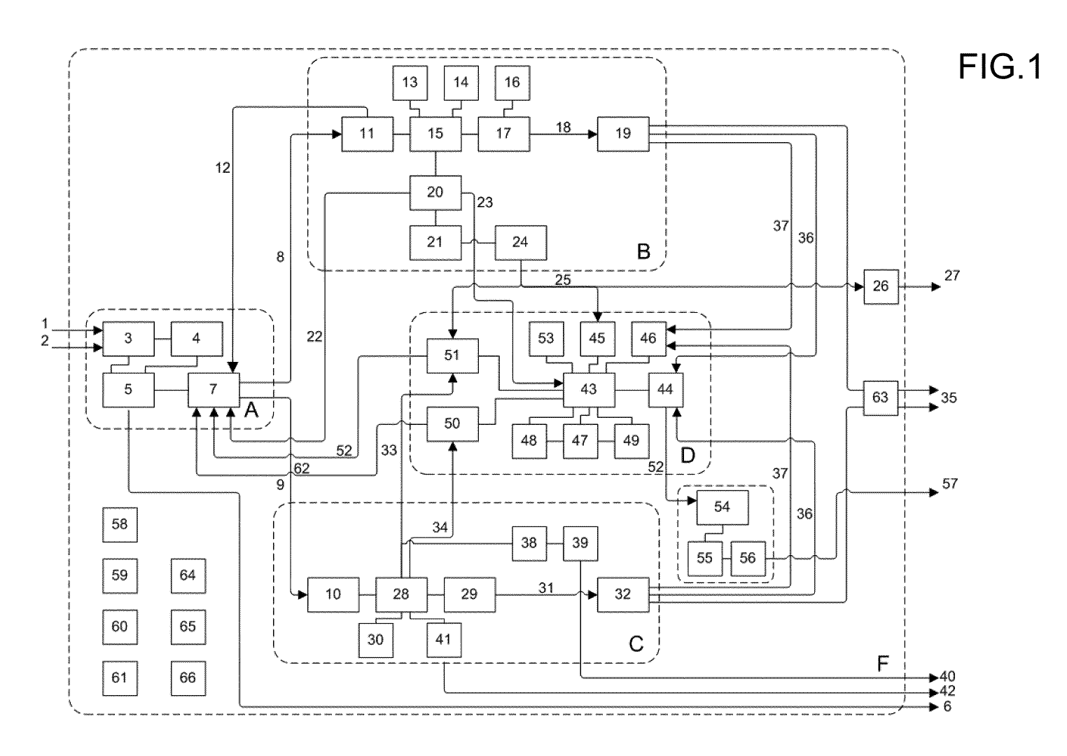

The integrated technological system of the invention is composed of integrated functional units arranged in series and parallel suitable for the performance of the subsequent operational phases required by the process as indicated in FIG. 1, maximising compactness, operational flexibility, production speed without intermediate stockings, with optimisation of the inter-exchange of the products of the functional units involved. It operates according to a principle of “just in time” of the operational phases that eliminates the criticalities connected to the typical phases of management and stocking of the digestate and the conversion into compost that require long times (up to 75 days for maturation with sanitation cycles of 15 days and turning every 15 days), large infrastructures and logistic spaces as well as risks correlated to pathogenic effects (bacteria, fungi, viruses etc.) and smelly emissions with nauseating odours.

This object is achieved thanks to a process formalised with specific standard operational procedures and a system having the characteristics listed in one or more of the claims to follow.

The process of the invention provides the advantages of being:
I. A sustainable solution (Smart Solution) for the co-production of bioenergy and products from the integrated conversion of biomasses and wastes including sludge from the purification of the sewage waters of the community with an advantage for the community itself (converting a problem into a unique opportunity) targeting toward a virtuous, safe, transparent and socially acceptable cycle, preventing smelly emissions and the NIMBY syndrome;
II. Sustainable solution (Smart Size) also for the small engineering size based upon a system classifiable as a “non incineration technology” fed by 5,000-10,000 t/y, but preferably about 25,000 t/y of wastes (INPUT). For example, if the input is made of unsorted municipal wastes, this amount refers to a community of about 50,000 inhabitants (referred to a production of 550 kg/y per inhabitant), whereas if the input is Organic Fraction of Municipal Solid Waste, it refers to a community of about 250,000 inhabitants (referred to a production of 100 kg/y per inhabitant) whereas if the input is made of biomasses with various energy content “cocktail” it is possible to reduce the collection basin up to a community of 20,000-25,000 inhabitants.
III. Opportunity for co-production (Smart Economy) of bioenergy and products (OUTPUT) that, when referred to a feeding with the equivalent of 1 t of municipal wastes with a typical composition (reference humidity about 30%, LHV about 10 MJ/Kg) is capable of producing bioenergy (electric up to about 800 kWh, thermal energy up to about 1,000 kWh) and products consisting mainly in high value agricultural and food products cultivated in open fields and/or in intensified greenhouses, primary lignocellulosic and algal biomasses and/or residual biomasses from functional unit D “Smart Farm”, or possibly biochar and biofuel.
   IV. Technical-operational sustainability with the surprising integration of technologies and processes (BAT/BEP) in terms of efficiency, flexibility, reliability and synergy among the Functional Units of the system;
      A—Reception of the initial materials and press-extrusion with separation of the organic liquid fraction (OLF) and energetic dry fraction (EDF),
      B—Biochemical conversion with co-generation from biogas (operational availability 7,500 h/y),
      C—Thermo-chemical conversion with co-generation from syngas (operational availability Z 7,500 h/y),
      D—Smart Farm with phytodepuration and cultivation in open fields and/or intensified and air-conditioned greenhouses for the production and valorisation of by-products (thermal energy, water, biochar, nutrients such as CO₂, NOx, ashes etc.) to trigger and self-feeding the local virtuous cycle and fight dryness by means of the recovery of water resources, as well as the selective cultivation targeted toward phytoremediation of contaminated soils (heavy metals, persistent organic products etc.).
      E—Production of biofuel (optional) to feed the municipal waste collection vehicles, agricultural machinery and/or urban mobility vehicles,
      F—Smart Dome—functional and protection as well as local and remote monitoring and supervision architectural structure.
   V. Economic sustainability of Green Economy with competitive market rates, coverage in terms of safe and continuous availability of initial materials to be converted and stability of the income deriving from the delivery and the subsidised rates provided by Laws at long term (up to 15-20 years for electric energy);
   VI. Financial sustainability of the single Functional Units (Payback 36-48 months) and reduced initial investment per ton of material converted with respect to a typical advanced incinerator, also classifiable as separate and independent “Business units” preferably implemented through Project Financing instruments since they are supported by strong Business Plans and All Risk Insurance Coverages;
   VII. Environmental sustainability provided by the proximity between production and conversion of the wastes (Zero km paradigm), minimising the logistics as well as minimising, if not totally eliminating, non-recoverable residues such as ash inerts (Zero waste paradigm) and the reduction of direct emissions into the atmosphere when the biochar produced is used as amendment (1 kg of biochar ≈3 kg CO_{2equivalent} sequestered (CO₂ negative paradigm);
   VIII. Green landscape sustainability with agricultural buffer zone “Smart Farm” for the visual, functional and anti-wind protection of the systems of a higher strategic value—“Smart Dome” to fight dryness, the effect of possible erosion and desertification;
   IX. Social sustainability with the creation of high profile jobs for young people (Green Jobs) for the Smart Farm and the management of the consortium of Functional Units with E-learning and E-maintenance technologies and quality assurance procedures (ISO 9001, ISO 14001, etc.) ensuring the functional reliability and transparency toward stakeholders, various investors and All Risks Insurance Coverages;
   X. Sustainable and continuous innovation with the intensification of processes realised based upon dehumidification by means of high pressure hot air on the energetic dry fraction (functional unit A), hyper-dynamic selective cavitation (functional units B, D, E), functionalised nanosponges (functional units B, D, E) solar concentration (functional unit C), valorisation of the water cycle for the production of drinking water and/or water for the controlled irrigation of open field and/or intensified greenhouse cultivations (functional unit D), enrichment with nutrients such as CO₂ for the photo-chemical conversion (functional unit D), semi-transparent thin film photovoltaic panels (functional unit D), artificial lighting, electro-bio-stimulation (functional unit D), intensification of the Fischer-Tropsch process for the production of liquid biofuel with ultra-compact micro channel technology (functional unit E) delignification and fermentation for the production of third generation bio-ethanol bioliquid (functional unit E).

The system is focused in particular for different operational scenarios both at climatic type (temperate or extreme cold/hot) and in zones with a high risk for erosion and/or desertification.

Functional unit A is essentially composed, by way of non-limiting example, of what is indicated in functional unit A of FIG. 1 and provides one or more of the following key functions and/or processes: I—Reception 3 of the load of initial materials 1 and 2 with weighing, on-line inspection of the load by means of reception multi-detector portal (i.e. equipped with radioactivity sensors, thermographic and visual profile, explosivity screening with profile of elements etc.), sampling for visual and/or instrumental inspection; II—reception of the load with relevant technical-administrative procedures; III—stocking in confined and protected area 4 limited for medium size volumes, corresponding to 3-5 days; IV—pre-conditioning of the material by crushing and selective separation of ferrous and non-ferrous metals, glass and inerts 5 possibly present, V—press-extrusion 7 at high pressure preferably with pre-dehumidification by means of high pressure hot air of the energetic dry fraction EDF; VI—separation of plastics 11, VII—supply of the liquid organic fraction 8 to functional unit B with the biochemical conversion system for biogas 15 and energetic dry fraction (EDF) 9 to functional unit C with the system for the thermo-chemical conversion for syngas 28; VIII—recovery from functional unit B of digestate solid 22 for press-extrusion 7 for the thermo-chemical conversion in functional unit C avoiding the aerobic maturation phase typical of the composting systems with the associated odour generation and pathogenic agents criticalities; IX—recovery from functional unit C of inerts, ashes 33 and residual solids; X—transfer of the ferrous and non-ferrous metals, inerts 6 to third parties authorised for the specific recovery of said materials; XI—vitrification of residual solids 39 from functional unit C and valorisation as a product; XII—use of functionalised nanosponges 14 with disinfecting agents for bacteria, fungi and viruses.

Functional unit B is essentially composed of, by way of non-limiting example, of what is indicated in functional unit A of FIG. 1 and provides one or more of the following key functions and/or processes: I—reception of the organic liquid fraction (OLF) 8 from functional unit A; II—biochemical conversion 15 of the organic liquid fraction (OLF) 8 with production of biogas 18 capable of ensuring a high operational availability of at least 7,500 h/y for a life cycle of at least 15-20 years under the expected climatic conditions; III—production and sale of electric energy 35 by means of biogas powered co-generation 19 for supply to the grid through the electric infrastructure (LV/MV cabin) and connection to Smart Grid 63; IV—production of thermal energy 36 and CO₂ 37 for supply to functional unit D and/or third party users; V—process intensification up to 20% increment of the efficiency and reduction of the biochemical conversion 15 by means of particles disintegration and sterilisation by hyper-dynamic selective cavitation 13 (HDSC) with formation of high pressure and high specific energy micro-bubbles during the pre-digestion homogenisation phase; VI—process intensification by means of functionalised nanosponges 14 for the inoculation in the bio-digester of selected strains of bacteria and/or enzymes which were selected and engineered to optimise the methanisation in terms of enhanced production, reduction of times and volumes of the reactors with a subsequent reduction of investment and operational costs, as well as the inoculation of digestate 22 in the process sewage waters to be treated with multi-spectrum biogenic disinfectant agents (anti-bacteria, anti-fungi, anti-virus); VII—suppression of the foam by means of sound-acoustic apparatus 16; VIII—purification of biogas 18; IX—removal of the solid from digestate 22 and plastics 12; X—provision of the solid from digestate 22 and plastics 12 to functional unit A for press-extrusion 7; XI—production of quality digestate 23 to be used as agricultural amendment; XII—supply of process water 25 from water/solid separation from digestate 20 to functional unit D for phytopurification 51 by means of multi-spectrum biogenic disinfecting agent (anti-bacteria, anti-fungi, anti-virus) possibly after sterilisation and degradation treatment of undesirable organic compounds preferably with intensification apparatus by means of cavitation 13; XII—possible production of drinking water 27 by means of water treatment plant 26.

Functional unit C is essentially composed, by way of non-limiting example, of what is indicated in functional unit C of FIG. 1 and provides one or more of the following key functions and/or processes: I—reception of the energetic dry fraction (EDF) 9 (including the solid from digestate 22) from functional unit A; II—thermo-chemical conversion of the energetic dry section (EDF) 9 with production of syngas 31 capable of ensuring a high operational availability of at least 7,500 h/y for a life cycle of at least 15-20 years under the expected climatic conditions; III—production and sale of electric energy 35 by means of syngas powered co-generation 32 for supply to the grid through the electric infrastructure (LV/MV cabin) and connection to Smart Grid 63; IV—production of thermal energy 36 and CO₂ 37 for supply to functional unit D and/or third party users; V—process intensification by means of an effective system for the removal and energetic valorisation of TARs; VI—thermal recovery from cooling syngas 32 to dry the feeding material to the thermo-chemical conversion for syngas 28 and/or supply to functional unit D as well as supply of drying hot water 24 to the same functional unit D; VII—energy supply for heating at high temperature of thermally conductive bodies by means of apparatus with solar concentration panels 30 for the conversion of carbonaceous matrices in thermo-chemical system 28; VIII—production of biochar 34 from lignocellulosic and/or algal primary biomass for use in functional unit D “Smart Farm”; IX—treatment of ashes 33 with recovery of higher added value materials by means of extraction of metals from ashes 38 and subsequent treatment process by means of vitrification apparatus of residual solids 39 or disposal of inert material 40; X production of advanced materials such as multi wall nanotubes 42 from the conversion of residual polymeric material under hyper-dynamic conditions 41; XI—production of nutrients such as CO₂, nitrogenous and sulphurised compounds 37 and ashes 33 for the process intensification of the production of algal biomass 52 in functional unit D.

Functional unit D is essentially composed, by way of non-limiting example, of what is indicated in functional unit D of FIG. 1 and provides one or more of the following key functions and/or processes: I—natural valorisation of water resources 25 coming from functional units B and C for phytopurification 51 also under extreme cold and windy climate conditions where an anti-freezing function is required (i.e. Mongolia, Russia etc.) with the production of high yield primary biomass 62 for possible feeding of functional unit A and to trigger a virtuous cycle; II—optimised irrigation by treated water 25 for cultivation in open fields and/or advanced intensified greenhouses 44 by means of irrigation water distribution apparatus 45; III—valorisation of thermal energy 36 for the heating/cooling of advanced intensified greenhouses 44; IV—cultivation in advanced intensified greenhouses 44 by means of a distribution apparatus for gaseous matrix enriched with nutrients such as CO₂, nitrogenous and sulphurised compounds 37 for the intensification of process 46 coming from functional unit B with the production of high value food-grade agricultural and food products for the local community; V—cultivation of algal biomass 52 by means of a distribution apparatus for gaseous matrix enriched with nutrients such as CO₂ and/or nitrogenous and sulphurised compounds 36 for the intensification of process 46 coming from functional unit C (non-food grade) and an additional purification process of the gaseous emissions from the gas co-generation; VI—use of biochar 34 (upon request) coming from functional unit C fed with primary biomass deriving from cultivation in unit D (for example genus Tamarix in the family Tamaricaceae with yields 400-500% higher than those from poplar); VII—process intensification for cultivation in open fields and/or advanced intensified greenhouses 44 by means of functionalised nanosponges to dispense microelement, nutrients 53 and multi-spectrum biogenic disinfectants; VIII—intensification process for cultivation in intensified greenhouses 44 by means of an apparatuses for cycles of high efficiency natural and/or artificial lighting 48 for multi-spectrum greenhouses preferably powered by semi-transparent thin-film photovoltaic system 47 with accumulation of electric energy; IX—intensification process for cultivation in intensified greenhouses 44 by means of an apparatuses for cycles of high efficiency natural and/or artificial lighting 48 preferably powered by semi-transparent thin-film photovoltaic system 47 with accumulation of electric energy; X—intercrops production cultivation 62 for selected biomass targeted toward agricultural and food and/or bio-energetic applications to feed functional unit A for the sustainability of the virtuous cycle or selected biomass for phytoremediation of contaminated soils.

The Smart Farm constitutes a green zone for agricultural use functioning as a buffer zone for the systems where the products for bio-energetic use for the virtuous cycle and high added value products for the community and the market are produced. Likewise the Smart Farm can be a place where young people with a high professionalism are employed (Green Jobs) for a long term sustainable development of the territory.

Functional unit E is essentially composed, by way of non-limiting example, of what is indicated in functional unit E of FIG. 1 and provides one or more of the following key functions and/or processes: I—stocking of biogas 18 and/or syngas 31 coming from functional unit B and/or C: II—treatment, compression and possibly specific enrichment for application in gas-powered engines for traction equipment for a sustainable mobility—“Smart Mobility”; III—distribution for applications with agricultural tractors and/or vehicles for the collection and management of municipal wastes, IV—production of liquid fuels with—Gas to Liquid GtL processes—new generation Fischer-Tropsch (i. e. micro-channel technology etc.); V—production of third generation bio-fuels (bio-ethanol) deriving from the enzymatic conversion of algal and/or lignocellulosic biomass obtained from intercrops production 62 in functional unit D previously treated with an intensification apparatus by means of hyper-dynamic selective cavitation 13 and fermentation with functionalised nanosponges for the dispensing of selected and engineered strains of bacteria and/or enzymes.

Functional unit F is essentially composed, by way of non-limiting example, of what is indicated in functional unit F of FIG. 1 and provides one or more of the following key functions and/or processes: I—mitigation of the visual impact by means of a green landscape (Green Land); II—protection of the systems from the action of extreme climatic events (i.e. strong winds etc.); III—phytodepuration 51 with selective vegetable species located on the slope constituted by the soil created on top of a bio-membrane conveying the purified process water from units B and C used for irrigation in unit D; IV—external protection from winds for intercrops production 62 and in advanced intensified greenhouses 44 by means of the dedicated cultivation of vegetable barriers (up to 10 metres high) optimised according to the anemometric profile resisting to the local climatic conditions; V—energy production by means of photovoltaic system 60 installed on top of tension protection structure 59 and/or energy collection system using an apparatus with solar concentration panels 30 for high temperature thermo-chemical conversion 28 in functional unit C; VI—access passages 65 to the arena where the priority functional units are located; VII—connection to the electric power network by means of the electric infrastructure (LV/MV cabin) and connection to Smart Grid 63; VIII—semantic interface for diagnostic and prognostic coverage for the effective management of the life cycle of the systems, apparatuses and strategic components of the system; IX—production of drinking water 27 by means of water purification apparatus 26.

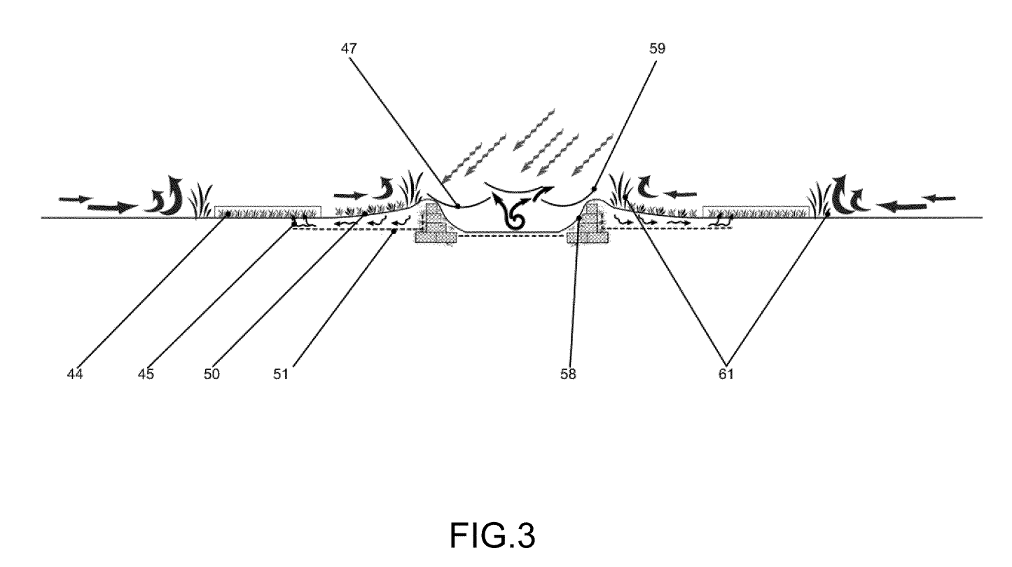

The Smart Dome has a round or polygonal layout of a size suitable to include functional units A, B, C and F anticipated and has an aerodynamic shape that in the vertical section, as shown in FIG. 3, has an hyperbolic profile optimised for the wind flows deriving from local anemometric conditions. The primary protection structure of the Smart Dome is preferably limited by gabions filled with locally available materials with a low environmental impact.

### Example No. 1

This example is focused upon the production of bio-energy and products from the conversion of municipal solid wastes (MSW) and provides the simplest, most economical, efficient, flexible and safe BAT/BEP solution for a typical city community of about 50,000 inhabitants (ref 540 kg/inh per year). The example solves the typical criticalities deriving from the typologies of selected collection and not better underlined in Examples 2 and 3. The solution is provided by an integrated system having a conversion capacity of 25,000 t/y fed by Unsorted Municipal Solid Wastes (UMSW) that shows the typical composition indicated in Table 1 with a calorific value of reference equivalent to 10,500 KJ/kg and humidity equivalent to about 33% in weight.

TABLE 1
Composition of UMSW (Source of report: processing of a
methodology finalised toward the determination of the calorific
value of unsorted municipal solid waste delivered in the
Province of Turin - March 2011 - IPLA Turin)

| Component | [% p/p] | Component | [% p/p] |
|---|---|---|---|
| Undersieve <20 mm | 6.97 | Wood | 1.17 |
| Organic | 22.12 | Natural textiles | 3.27 |
| Green | 2.27 | Other textiles | 1.39 |
| Plastic film | 6.82 | Total textiles | 4.66 |
| Other plastics | 7.08 | Skins and leather | 0.55 |
| Plastic containers | 2.51 | Glass | 6.59 |
| Total plastic fraction | 16.41 | Other inerts | 3.55 |
| Recyclable paper | 6.88 | Alluminium | 0.81 |
| Other paper | 7.49 | Ferrous metals | 2.33 |
| Flat cardboard | 3.06 | Non-ferrous metals | 0.78 |
| Corrugated cardboard | 2.56 | Torch batteries | 0.12 |
| Tot. paper fraction | 20.44 | Drugs | 0.11 |
| Diapers | 7.18 | Other dangerous waste | 0.06 |
| Foil lined paper | 3.19 | | |
| Foil lined plastic | 0.65 | | |
| Foil lined alluminium | 0.04 | | |
| Tot. foil lined packages | 3.88 | | |

TABLE 2
Energy data of UMSW in the Province of Turin
Province of Turin (data 2009)

| LHV dry | LHV as is | Humidity |
|---|---|---|
| kJ/kg | kJ/kg | % |
| 16,793 | 10,480 | 32.72 |

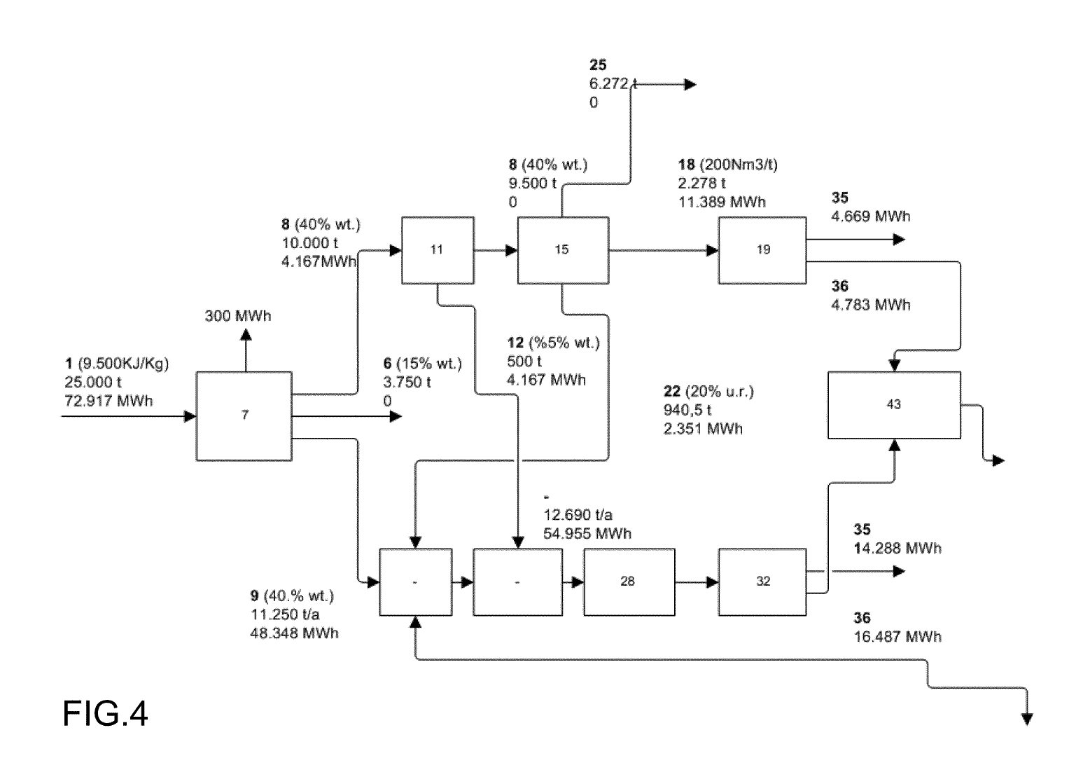

The conversion of the UMSW is carried out in functional units A, B, C, D, F which operate in an integrated manner as outlined in FIG. 4 that lists the mass/energy balance. The UMSW is received in functional unit A and subject to a high pressure press-extrusion (with a low specific electrical consumption equivalent to 12 kWh/t of UMSW) by an apparatus as described in Patent Application no. ITTO20100192 to produce two fractions: organic liquid (OLF) equivalent to about 40% and one equivalent to about 60% thet is sub-divided into energetic dry fraction (EDF) (about 45%) and metal materials (ferrous and non-ferrous), glass and inerts (about 15%). From the OLF the aliquot of residual plastic material it contains equivalent to about 500 t/y (about 5% in weight of the OLF) is extracted and it is sent to functional unit C for the hyper-dynamic thermo-chemical catalytic conversion (HTCC) as described in WO2012/085880. The OLF that amounts to 9,500 t/y is sent to functional unit B for the bio-chemical conversion to produce biogas in an advanced bio-converter as described in Patent Application no. ITTO20080394 with an efficiency of about 200 Nm³/t of OLF that is 1,898,100 Nm³/y with a calorific value equivalent to 6 kWh/Nm³ (composition 60% of CH₄) capable of producing 4,669 MWh/y electric (electric efficiency gas engine Jenbacher J312 equivalent to about 41%) (power generated 543 kWe) to be supplied to the Smart Grid through the electric cabin and thermal energy equivalent to 4,783 MWh/a (efficiency about 42%) to be valorised in functional unit D Smart Farm. The operational availability of the biogas powered co-generator is at least 8,600 hours per year. Functional unit B produces also digestate that after dehumidification with a resulting humidity equivalent to 20% amounts to 940 t/y to be sent to functional unit C and water equivalent to 6,272 t/y (equivalent to about 25% of the initial MSW) to be sent, after treatment, to functional unit D for valorisation by means of phytodepuration for irrigation. The metallic materials (ferrous and non-ferrous), the recyclable inert products such as glass are delivered to functional unit A to be forwarded to the salvage chain, for the valorisation and/or disposal by authorised third parties. The EDF is separated from its aliquot of ferrous and non-ferrous metals, inert products such as glass and consisting of about 15% that is 3,750 t/y. Then the EDF consisting of material coming from functional unit A (initial material+plastics+digestate) amounts to 12,690 t/y. The EDF after drying with the recovery of heat from functional unit B, which has a calorific power of about 15,500 kJ/kg with a humidity of about 10% in weight, is undergoing a thermo-chemical conversion in functional unit C to produce electric energy and heat. The annual production of electric energy to be delivered to the Smart Grid through the electric cabin is equivalent to 14,288 MWh/y (electric efficiency of the syngas system+2 co-generators GE Jenbacher J320 equivalent to 26%) with an installed power of the co-generator of about 1,905 kW, whereas the thermal production amounts to about 16,487 MWh/y considering the operational availability of at least 7,500 hours per year. The solids deriving from the thermo-chemical conversion are essentially ashes equivalent to about 337 t/y (3% in weight with respect to the EDF) and solids and inerts correlated to the thermo-chemical process equivalent to about 506 t/y (4.5% in weight with respect to the EDF) to be delivered to functional unit A to be forwarded to the salvage chain by means of vitrification and then converted into a positive value added product for the construction sector with a market economic return. A fraction of the ashes finds an application as nutrient in the cultivation of algal biomass. In fact, the Zero Waste condition is achieved since all the initial materials are converted into bio-energies and products and the ferrous and non-ferrous, materials, inerts, ashes and water find their functional and/or economical revaluation.

The thermal energy is valorised in functional units C, D and F for thermal recovery and air-conditioning for the cultivation in intensified greenhouses, whereas the gas flow enriched with CO₂ (production of CO₂ equivalent to 630 g/KWhe—source ENEA) deriving from the fumes previously treated and equivalent to about 11,943 t/y of CO₂ (equivalent to 0.44 t/t_{MSW}) is sent to the greenhouses of functional unit D as nutrient for the intensification of the photo-synthetic process in the production of primary and/or algal biomass. The thermal energy, the water resource, the nutrients such as ashes and CO₂ feed a Smart Farm equipped with a phytodepuration system of the process waters generated that amount to about 6,312 t/y, which allow the growth of selected vegetable species for the short rotation intensive cultivation of the species Tamarix with a yield up to an above 50 t/year per hectare in open fields in the zone nearby the systems zone and that constitutes the intercrops area. The zone occupied by the Smart Dome and the Smart Farm has a surface of about 5 hectares, where functional units A, B, C are arranged in a technological area of about 5,000 m². The types of process characterised by a high processing speed prevent the criticalities created by smelly emissions together with compactness and the intrinsic confinement of the process zones make the system neutral and “environmentally friendly”. Outside the zone occupied by functional units A, B, and C, the buffer zone is located for the visual, functional and anti-wind protection as well as the enhancement of the green landscape aspect which is a key factor for the environmental sustainability and social acceptance. An educational and recreational path in the green with pause points and illustrative and/or inter-active totems seeks the involvement of the different generations at different levels (school, family, social). The Smart Farm, beyond the intercrops zone, provides for the radial presence of the above said intensified greenhouses with a semi-circular section (equivalent to 3 m radius) as modular sections made of advanced semi-transparent polymeric material for the cultivation of high added value products such as for example flowers and algal biomass in advanced photo-bio-reactors. In the intensified greenhouses is always available the water purified by the phytodepuration system possibly integrated by external sources for the dedicated cultivations. Also, the air-conditioned greenhouses are a confined ambient for the use of the CO₂ enriched gaseous flow to intensify the production of the said cultivations. An electrical systems provides cycles of artificial lighting inside the greenhouses. In case local norms (i. E. European Directive 98/2008) prescribe as a priority factor the recovery and recycling of materials, the quality digestate can be used as agricultural amendment. In this instance there is an immediate marginal reduction of the production of energy against a correlated enhanced production of products and biomasses. For each ton of UMSW as is of reference, having for example a LHV of 10,500 KJ/kg bioenergy equivalent to 758 KWh electric is produced (total efficiency 26.0%) as well as 851 KWh thermal (total efficiency 29.2%). It is possible to scientifically anticipate that the performances can be enhanced at least 10-15% depending upon the typology of the wastes currently collected and the intensification and optimisation of the processes. A particular importance is the fact that these energy resources available provide the production of products with a high market value computable in at least 250 t/year. The water resource is recovered and valorised and out of about 8,250 t/y contained in the MSW as is (33% water on the as is) 6,272 t/y are recovered that is 76% in weight of the input water. This pragmatic solution in the political strategic scenarios offers the highest simplification and lower costs for the community of citizens taking advantage of all the benefits provided by the collection of unsorted wastes, that is the elimination of different containers and bags, with a lower visual, infrastructural and logistic impact, with different types of systems for the subsequent conversion, but most of all for the maximum valorisation of the bioenergy and products resources with the dynamic coverage at short, medium and long term according to the Guidelines at strategic, normative and political level.

The net investment financial requirement is about 20,000,000.00 [symbol] corresponding to about 800.00 [symbol] /t capacity. When simulating the scenario with the said characteristics in a provincial context in Turin, it is possible to convert and valorise 567,057 t/y that still represent the UMSWs (datum 2010) in electric energy equivalent to 430,005 MWh/y and thermal energy equivalent to 482,448 equivalent to a thermo electrical cogeneration station of about 55 MW of electric power (operational availability 7,800 hours/y). This example is a dynamic and flexible response to the evolution of the scenarios in which sorted collection does not and cannot reach in future the only theoretical target of 100%.

The application of this invention is effective also in a much more heterogeneous Italian scenario in which 32,000,000 t/y are produced (source ISPRA 2009) and in which sorted collection is far from reaching satisfactory levels. Similarly the system is able to satisfy the requirements of different global operational scenarios also under extreme climate conditions (cold/hot) as well as fighting phenomena of poverty, dryness and desertification also when external water and energy resources are scarce. The conversion solution into bioenergy and products of UMSWs provides an answer to what comes out from the comparative analysis of the best practices in Europe where it is demonstrated how a high level of energy recovery is necessary to abate the squanders correlated with the delivery to landfills that is the total energetic loss accompanied by the increment of environmental criticalities. Additional and decisive benefits derive from the emission of CO₂, with respect to the direct consumption of fossil fuels, besides the possibility of offering progressive reductions of the rates in total terms for individual citizens, being the costs for the management of MSWs almost totally covered by tariffs (or local taxes) resting on individual families.

This example demonstrates the surprising advantages offered by this virtuous cycle which is sustainable under the technical, energetic, economic, financial, environmental, landscape and social profiles as well as toward social acceptability by the communities and for the stakeholders.

### Example No. 2

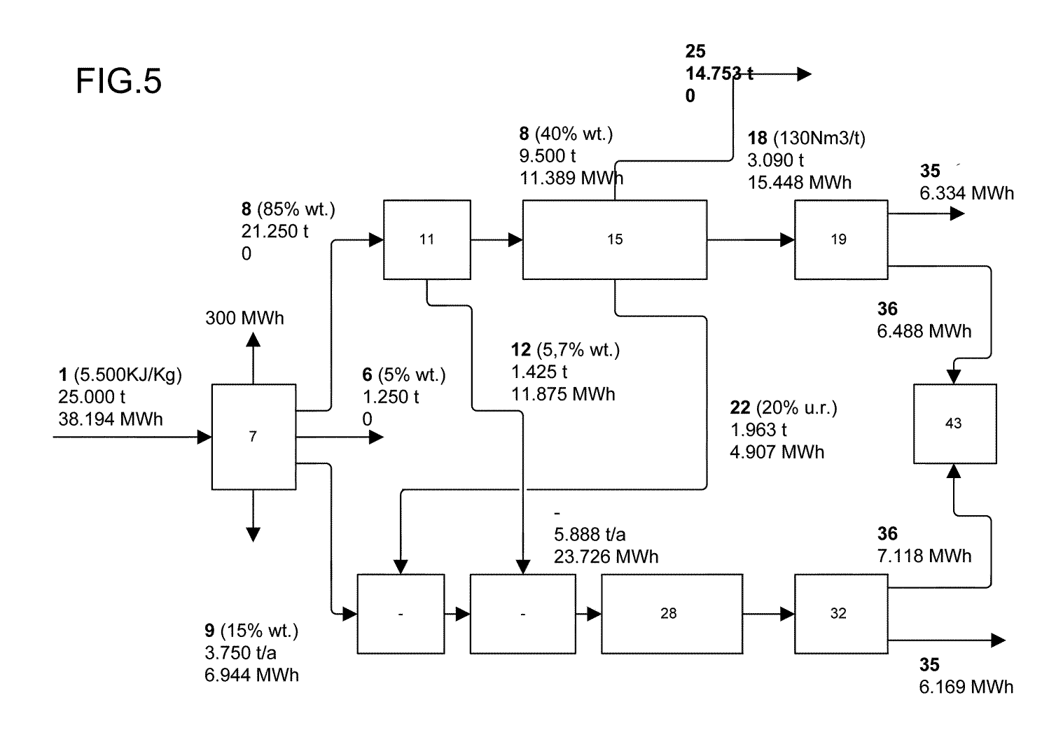

This example is focused upon the production of bio-energy and products from the conversion of the organic fraction of municipal solid wastes (OFMSW) deriving from the sorted collection of MSWs and provides the simplest, most economical, efficient, flexible and safe BAT/BEP solution for a typical city community of about 330,000 inhabitants (ref 75 kg/inh per year). The solution is provided by an integrated system with the conversion capacity of 25,000 t/y fed by OFMSWs having the following typical composition: organic fraction 89.30%, plastics 5.70%, ferrous and non-ferrous metals 2%, glass and inerts 3% with a calorific power of reference equivalent to about 5,500 KJ/Kg and a humidity equivalent to about 65% in weight. The system for the conversion of the initial material has the same engineering configuration indicated in Example 1 demonstrating the surprising operational flexibility being able to convert effectively with easy adaptations both UMSWs and OFMSWs. The initial OFMSW material is received in functional unit A and subject to a high pressure press-extrusion (with a low specific electrical consumption equivalent to 7 kWh/t of OFMSW) to produce two fractions: organic liquid equivalent to about 85% and solid (EDF) equivalent to 15% in weight. The OLF and EDF fractions are converted in functional units B, C, D, E as described in example 1 with the mass/energy balances indicated in FIG. 5. In case local norms (i. E. European Directive 98/2008) prescribe as a priority factor the recovery and recycling of materials, the quality digestate can be used as agricultural amendment. In this instance there is an immediate marginal reduction of the production of energy against a correlated enhanced production of products and biomasses. The criticalities correlated with the production of compost from OFMSW are solved by means of aerobic cells (sanitation cycle of 15 days at 70° C. and subsequent maturation cycle of about 60 days to prevent pathogenic risks), simplifying the logistics, conversion times, eliminating smelly emissions and maximising the energetic valorisation of the material making the OFMSW. In particular, this situation solves the criticalities correlated to the missed sale of the compost that has a market value near zero Euro due both to a lack of demand and, often, to the nonconformity of the compost for agricultural and food application (concentration of heavy metals, plastics, glass etc.). In this manner all the biomass made by the fraction of lignocellulosic material that otherwise would be used to formulate the compost (up to 30% in weight) can be effectively valorised for energy. In relation with the conversion system of 25,000 t/y of OFMSWs 12,502 MWh/y of electric energy are produced (with output power of 736 KWe from biogas powered co-generator and 823 KWe from syngas co-generator), whereas the thermal energy is equivalent to 13,606 MWh/y. For each ton of OFMSW as is of reference for example having a LHV of 5,500 KJ 7 kg bioenergy is produced equivalent to 500 KWh electric (total efficiencies 32.7%) and 544 KWh thermal (total efficiencies 35.6%). The water resource is recovered and valorised and out of about 16,250 t/y contained in the OFMSW as is (65% water on the as is) 14,753 t/y are recovered that is 91% in weight of the input water.

### Example No. 3

This example is focused toward the co-production of bioenergy and products from the conversion of municipal solid wastes (MSW) in the typical territorial scenario in which incinerators are present in the sorting and collection chain as well as systems for the production of compost from OFMSWs, demonstrating the synergic integration capability.

The scenario for example for the Province of Turin points out with the data relative to the year 2010 in which the production of Municipal Solid Waste reaches 567,057 t/y (246 kg/inhab per year) whereas the waste from sorted collection is equivalent to 560,365 t/y (243 kg/inhab per year) for a total collection of 1,127,422 t/y (equivalent to 490 kg/inhab per year) with a percentage of sorted collection reached 49.7%. The organic fraction amounts to 142,293 t/y (equivalent to 12.6% of the total MSW) whereas grass cuttings and vines from pruning (residual biomass) reach 53,568 t/y (equivalent to 4.8% of the total MSW). The latter components make up the Organic Fraction of Municipal Solid Waste (OFMSW). The conversion of OFMSW as indicated in Example 2, currently shows criticalities in terms of: capacity of conversion, quality of the compost for agri-food use, demand and relevant market value practically nil. The subject solution, in fact, can make a complementary opportunity for the current project that shall be running in January 2014 (development delay equivalent to about 48 months) in Turin referred to the new incinerator TRM—Trattamento Rifiuti Metropolitano—Metropolitan Waste Treatment—www.trm.to.it—for the combustion of 421,000 t/y of Municipal Solid Waste (MSW) residual from sorted collection and special waste comparable to municipal wastes. It must be noted that the TRM plant does not include the collection of unsorted municipal waste nor least of all, the organic fraction (OFMSW) plus biomasses deriving from sorted collection. The net financial requirement is about 503,000,000.00 [symbol] (Project Financing data 2008) corresponding to 1,195.00 [symbol] /t capacity (referred estimated data 2008 with the final balance data 2013 that includes a substantial increment). The TMR plant fed with materials carried by 40 lorries and 1 train with 16 carriages per day converts this material equivalent to 421,000 t/y into electric energy equivalent to 350,000 MWh/a (efficiency 21.8%) and thermal 170,000 MWh/y (operational availability 7800 h/y). The electric power of the plant is equivalent to about 45 MWe. To ensure the minimal operational conditions the installation of auxiliary natural gas burners is planned for an estimated requirement of 1,600,000 Sm3/y (3.8 SM³_{natural gas}/t_{waste}).

The TMR incinerator has high landscape impacts due to large structures (i.e. a 120 m tall smokestack, 100,000 sq m of land occupied) and infrastrucures as well as significant environmental impacts due to the concentration in one single point of the conversion of MSW deriving from a very large collection basin. It is not absolutely negligeable the requirement for industrial water equivalent to 1,000,000 t/a (2.37 t_{water}/t_{waste}) and the production of residual solids is equivalent to 110,723 t/y (26.3% of the feeding waste) with the following typical ratio of composition: 210 g/Kg slag, 18.5 g/Kg ferrous, ashes 20 g/Kg, dusts 15 g/Kg. The average market conventional rates for traditional disposal of municipal wastes are referred to controlled landfills of municipal wastes at about 100.00 [symbol] /t (data 2011) for OFMSW at about 90.00 [symbol] /t (data 2011), for the dry fraction from sorted collection to be delivered to the TMR incinerator of Turin has been established in 2008 at 97.50 [symbol] /t (the delivery conventional rate shall have a substantial increment when the plant shall run in 2014). In comparative terms, the dry fraction from sorted collection to be delivered to the IREN incinerator in Parma has been established at 168.00 [symbol] /t (to be completed within 2012 and started in 2013).

The comparative Table 3 below summarizes the evaluation factors both in quantitative and qualitative terms between the various conversion technologies for Municipal Solid Wastes.

TABLE 3
comparison table

| | | | | | BioEnPro | |
|---|---|---|---|---|---|---|
| | | | | | combined with | Thermal |
| | | | Aerobic | Anaerobic | anaerobic | valorization |
| Pos | Parameter | BioEnPro | composting | composting | composting | (incineration) |
| 1 | Capacity (tpy) | 25,000 | 25,000 | 25,000 | 25,000 | 421,000 |
| 2 | Suitable for unsorted MSW | yes | no | no | no | no |
| 3 | Suitable for organic MSW fraction | yes | yes | yes | yes | no |
| 4 | Suitable for MSW with a prevailing dry fraction | yes | no | no | yes | yes |
| 5 | Specific capital costs (per ton of capacity) | [symbol] 800* | > [symbol] 250⁺ | > [symbol] 300⁺ | n/a | [symbol] 1,200** |
| | | | | | | [symbol] 1,465*** |
| 6 | Specific power production (kWh/ton)/ | 760/26 | — | — | — | — |
| | Efficiency (%) from unsorted MSW (LHV | | | | | |
| | 10,500 kJ/kg) | | | | | |
| 7 | Specific power production (kWh/ton)/ | 500/32.7 | — | 250/16.3 | 500/32.7 | — |
| | Efficiency (%) from organic MSW fraction (LHV | | | | | |
| | 5,500 kJ/kg) | | | | | |
| 8 | Specific power production (kWh/ton)/ | — | — | — | — | 666/21.9 |
| | Efficiency (%) after MSW sorting (LHV 11,000 | | | | | |
| | kJ/kg) | | | | | |
| 9 | Suitability of the exploitation of thermal energy | yes | — | no | yes | no**** |
| 10 | Savings of primary energy (toe/year)⁺⁺ | 3,545/2,338 | — | 1,168 | 2,338 | 65,450 |
| 11 | toe index⁺⁺⁺ | 0.251/0.131 | 0.131 | 0.131 | n/a | 0.263 |
| 12 | Production of bio-products | yes | no | yes | yes | no |
| 13 | Production of quality soil conditioner | yes | yes | yes | yes | no |
| 14 | Production of biochar | yes | no | no | yes | no |
| 15 | Production of biofuels | yes | no | yes | yes | no |
| 16 | Production of solid residues considered as | no | yes | yes | no | 26% |
| | waste (ton/ton_{waste}) | | | | | |
| 17 | Production of irrigation water from MSW | 25% | — | — | — | no |
| | (ton/ton_{waste}) | | | | | |
| 18 | Production of irrigation water from the organic | 60% | no | no | yes | — |
| | fraction of MSW (ton/ton_{waste}) | | | | | |
| 19 | Production of wastewater | no | yes | yes | no | yes |
| 20 | Odour emissions | Excellent | Poor | Average | Good | Good |
| 21 | Need for industrial water (ton/ton_{waste}) | no | yes | yes | no | 2.37 |
| 22 | Need for natural gas (Nm³/ton_{waste}) | no | no | no | no | 3.8 |
| 23 | Time required for construction (months) | 18 | 18 | 12 | 18 | 48 |
| 24 | Impact on infrastructures (roads, railways, etc.) | Excellent | Excellent | Excellent | Excellent | Poor |
| 25 | Technical reliability | Excellent | Poor | Good | Good | Excellent |
| 26 | Environmental benefit-cost ratio | Excellent | Average | Average | Good | Average |
| 27 | Industrial cost reasonableness analysis | Excellent | Good | Good | Excellent | Average |
| 28 | Workforce safety | Excellent | Good | Good | Good | Good |
| 29 | Visual impact | Excellent | Good | Good | Good | Average |
| 30 | Territorial and logistic impact | Excellent | Good | Good | Excellent | Poor |
| 31 | Effectiveness to counteract drought and | Excellent | Average | Average | Good | Poor |
| | desertification | | | | | |
| 32 | Operational flexibility in extreme environmental | Excellent | Average | Average | Excellent | Poor |
| | conditions | | | | | |

In relation with the evaluation factors indicated in the comparison table it is pointed out how the system subject of the invention satisfies the BAT/BEP requisites in terms of technical reliability, environmental cost/benefit ratio, reasonable costs at industrial level. Also, the high flexibility (waste from unsorted or sorted collection) and the high energetic efficiency (up to 32.7% in the case of OFMSW) of the system of the invention for the production of bioenergy and products deriving from the conversion of the various typologies of municipal wastes and biomasses generated by the community itself under various operational scenarios and with a low visual and environmental impact is demonstrated.

In particular it is evident that the efficiency is maintained also for small size systems (25,000 t/y or less) with reference to the State of the Art of the sector, thus it constitutes a valid solution also for small communities. The solution is economically competitive with an investment financial requirement per ton for the alternatives up to 40% lower with respect to incinerators and a conversion rate at least 10-15% lower with respect to the currently applied market rates. A virtuous circuit is created thanks to the complete valorisation of all the potential resources available of the waste (i.e. water for irrigation of dedicated cultivations, drinking water, CO₂ present in exhaust fumes, valorisation of nutrients and thermal energy etc.).

The examples list some illustrative, but not limiting, results of the possible operational scenarios for the conversion of municipal solid wastes as described in the field of application and in its scope.

1. Process for the recovery and the exploitation of a substratum of biomasses municipal wastes and/or carbonaceous matrices for the co-production of bioenergy and products by means of the synergic integration of different conversion processes and treatment of the emissions in closed-loop circuit encompassing the following phases:
using algal and lignocellulosic biomasses deriving from the exploitation of the by-products generated by primary conversion systems such as thermal and electric energy, digestate, biochar, water, CO₂ and nitrogenous and sulphurised compounds and ashes as nutrients for the production of vegetable organisms by means of intensifying the photosynthesis processes for the treatment of emissions and the sequestration of CO₂,
feeding the said substratum to a press-extrusion system, possibly provided of separation means, so that it is sub-divided into a liquid organic fraction and an energetic dry fraction,
using the said liquid organic fraction as feed for a biogas generation system by means of bacterial fermentation under anaerobic conditions,
using the said energetic dry fraction as feed for a synthesis gas generation system by means of a thermo-chemical conversion,
wetland bio-filtering water obtained as by-product from the biogas and syngas generation system, obtaining purified water and primary biomass,
using biogas and/or synthesis gas produced by a co-generation system and/or biofuels deriving from biogas and/or synthesis gas to feed vehicles.

2. Process according to claim 1, wherein the high-efficiency thermo-chemical conversion preferably under iper-dynamic and catalytic conditions by means of thermally conductive bodies, at a temperature included between 500° C. and 1000° C. with the production of synthesis gases and multi-wall carbon nanotubes is carried out inside a multi-stage rotary reactor contained by a fixed external shroud surrounding it.

3. Process according to claim 1, wherein the biochemical conversion, preferably in high-load bio-digestion systems, is carried out by intensifying the process by means of hyper-dynamic selective cavitation upstream a primary bio-digester and/or inoculation with functionalised nanosponges of selected strains which were engineered for the methanisation of bacteria and/or enzymes in the bio-digester.

4. Process according to claim 1, wherein the purified water obtained as by-product from the biogas and syngas generation system is used to irrigate open field cultivations and/or intensified greenhouses.

5. Process according to claim 1, using for the production of vegetable organisms one or more greenhouses, as well as functionalised nanosponges for the dispensing of microelements such as Fe, Zn and other magnetic metals, micronutrients, active principles and disinfectants.

6. Process according to claim 5, wherein said greenhouses are made with semi-transparent low-thermal transmittance polymeric material possibly incorporating a thin photovoltaic film.

7. Process according to claim 5, wherein the cultivation in said greenhouses is intensified by the synergic integration of one or more additional processes based upon electro bio-stimulation and cycles of artificial lighting.

8. Process according to claim 5, which is conducted also with the aid of photo-bioreactors for dedicated algal biomasses.

9. Process according to claim 1, wherein a buffer zone is present constituting the Smart Farm (functional unit D) for the social and green landscape sustainability as well as the protection of cultivation by wind barriers representing the countermeasure to fight dryness and desertification.

10. Process according to claim 1, wherein the functional units and the relevant centralised supervision and control centre are located in an architectural structure for green landscape sustainability as well as technological, designated as Smart Dome with a round or polygonal layout with an arena in the centre preferably covered by a tensioned protection structure which could incorporate a photovoltaic system and preferably limited by gabions for environmental sustainability and in the vertical section with anti-wind aerodynamic profile wherein in the slope a wetland filtration system is located dedicated to the waters recovered from functional units (A, B, C) with geo-membrane fed by a distributor from the top flowing by gravity in a reservoir and in the plain a functional unit D (Smart Farm) is located with access passages up to an anti-wind barrier and a cabin for the connection to the electric power network.

11. Process according to claim 1, wherein the digestate obtained as by-product from the biogas generation system is usable in functional unit D Smart Farm as good quality soil conditioner, whereas the amount in excess is converted into bio-energy and products by feeding the press-extrusion system.

12. Process according to claim 1, wherein ferrous and non-ferrous metals and inert are subject to a complete recovery, whereas ashes obtained as by-product by the synthesis gas system are usable by the Smart Farm as nutrient, whereas the amount in excess is subject to a vitrification process and the residual particle solids to obtain products for the construction or artistic market.

13. Process according to claim 1, wherein modular functional units are used.

14. Process according to claim 1, wherein a gas flow enriched with CO₂ deriving from fumes which are a by-product of primary conversion systems is sent to greenhouses as air-conditioner for the intensification of the photo-synthesis of cultivations, in particular flowers and algal biomass subjected to cycles of artificial lighting, wherein said greenhouses are thermally controlled.
